(19) United States
(12) Patent Application Publication
(10) Pub. No.: US 2024/0149057 A1
(43) Pub. Date: May 9, 2024

(54) DEVICE FOR TREATING USER'S SKIN USING PLASMA

(71) Applicant: JUBILEE INTERNATIONAL BIOMEDICAL CO., LTD., Taipei (TW)

(72) Inventors: HUI-FANG LI, Tainan (TW); YU-TING LIN, Tainan (TW); CHUN-HAO CHANG, Changhua County (TW); CHIH-TUNG LIU, Hsinchu (TW); CHUN-PING HSIAO, Taichung (TW); YU-PIN CHENG, New Taipei (TW)

(21) Appl. No.: 18/052,572

(22) Filed: Nov. 4, 2022

(51) Int. Cl. A61N 1/14; A61N 1/44; H05H 1/24

(52) U.S. Cl. CPC A61N 1/14; A61N 1/44; H05H 1/2437; H05H 1/2431; H05H 2245/34

(57) ABSTRACT

A device for treating a user's skin using plasma is provided. The device comprises a plasma generation assembly and a power supply. The plasma generation assembly comprises a discharge electrode including a first surface; a first dielectric material layer provided on the first surface of the discharge electrode and the first surface, a ground electrode surrounding the discharge electrode, and an insulation member spacing around the discharge electrode from the ground electrode. The power supply configured to apply power to the plasma generation assembly so that plasma is generated from the first surface of the discharge electrode to the ground electrode and between the first dielectric material layer and the user's skin.

### BACKGROUND OF THE INVENTION

### 1. Field of the Invention

The present invention relates to a portable and compact device making use of plasma, in particular to a portable and compact device for treating user's skin using plasma.

### 2. Description of Related Art

Plasma is the fourth fundamental state of matter after solid, liquid and gas. It is defined as “a quasi-neutral substance of charged and neutral particles which exhibits collective behavior” by couples of textbooks. Flame, lightning, polar light and sun are common forms of plasma state. Generally, the external energy is applied to a certain gas and free electrons in space are then accelerated, and thus, the free electrons impact the gas to produce more charged particles, active particles and free radicals so as to generate the plasma. To take a macroscope view, the gas still remains in an electrically neutral state, and the gas would emit light during the ionization.

Plasma is widely used in semiconductor manufacturing industry, such as plasma coating and plasma etching, and is also widely applied in consumer electronics, such as plasma flat screen TV. Besides, plasma has expended its application to the skin care market for the purpose of skin beauty, sterilization and/or treatment.

For instance, Patent document, WO2016/114504A1, provides a portable plasma skin improvement apparatus for improving a skin state by generating plasma through a dielectric barrier discharge at atmospheric pressure, as seen in FIG. 1. When the user touches the grounding portion (80) by hand, the projection (61) of the head (60) is in contact with the skin while the front cover (40) and rear cover (50) are gripped. Plasma is generated in the space between the surface of the upper plate (one component of the plasma generating module (30), which is not shown in FIG. 1) and the skin, but the plasma is not generated when the user does not contact the ground portion (80).

However, as one can observe from FIG. 1, the grounding portion (80) is arranged to be hold by user's hand, which generates a current loop passing through user's heart when the user uses the apparatus to touch the skin of the user's face and thus there are safety concerns, especially to those implanted with pacemakers.

Another patent document, US2018/0126183A1, provides a skin treating device (10), as seen in FIG. 2. In use, the driving device (18) is switched on by a user by means of the switch, and the skin interface (13) of the device (10) is positioned against or close to an area of skin to be sterilized, for example a wound. Once the driving device (18) is switched on, the driving device (18) generates a low voltage electrical signal which is transmitted to the primary winding coil of the transformer (25) via the plugs and sockets (33, 34). The transformer (25) changes the low voltage electrical signal into a higher voltage electrical signal which is applied between the main electrode (26) and the skin interface electrode (27). This creates electrical discharges which ionize air located between the main electrode (26) and the skin interface electrode (27) and generate non-thermal plasma at the skin interface electrode (27). The generated non-thermal plasma diffuses onto the skin, thereby disinfecting the skin.

However, as one can observe from FIG. 2, the electrical discharges, i.e. plasma, are generated between the main electrode (26) and the skin interface electrode (27). After the plasma is generated, the plasma diffuses a certain distance (labeled with “D” in FIG. 2) before reaching the user's skin. Therefore, in the patent document, US 2018/0126183 A1, active particles with short retention time in the plasma (e.g., hydroxyl groups with a retention time of only tens of nanoseconds) cannot be delivered to the surface (e.g., human skin) of the object to be treated, and the efficacy of treatment will be significantly limited.

Accordingly, it is desired to have another portable and compact device for treating user's skin using plasma without the aforementioned weaknesses.

### SUMMARY OF THE INVENTION

In view of the deficiency of the conventional devices for treating user's skin using plasma, the present application provides a device for treating user's skin using plasma designed to be safer and with high treatment efficiency.

In one aspect of the present invention, a device for treating a user's skin using plasma is provided, which comprises a plasma generation assembly, comprising a discharge electrode including a first surface; a first dielectric material layer provided on the first surface of the discharge electrode and the first surface facing toward the user's skin; a ground electrode surrounding the discharge electrode and being closer to the user's skin than the discharge electrode when the device is in use; and an insulation member spacing around the discharge electrode from the ground electrode; and a power supply configured to apply power to the plasma generation assembly so that plasma is generated from the first surface of the discharge electrode to the ground electrode and between the first dielectric material layer and the user's skin.

In another aspect of the present invention, a device for treating a user's skin using plasma is provided, which comprises a plasma generation assembly, comprising a ground electrode; a discharge electrode surrounding the ground electrode and being farther from the user's skin than the ground electrode when the device is in use; a first dielectric material layer provided on a first surface of the discharge electrode and the first surface facing toward the user's skin; and an insulation member spacing around the ground electrode from the discharge electrode; and a power supply configured to apply power to the plasma generation assembly so that plasma is generated from the first surface of the discharge electrode to the ground electrode and between the first dielectric material layer and the user's skin.

In yet another aspect of the present invention, a device for treating a user's skin using plasma is provided, which comprises a plasma generation assembly, comprising a discharge electrode; a first dielectric material layer provided on a first surface of the discharge electrode and the first surface facing toward a user's skin; a ground electrode adjacent to the discharge electrode and being closer to the user's skin than the discharge electrode when the device is in use; and an insulation member spacing the discharge electrode from the ground electrode; and a power supply configured to apply power to the plasma generation assembly so that plasma can be generated from the first surface of the discharge electrode to the ground electrode and between the first dielectric material layer and the user's skin.

In yet another aspect of the present invention, the insulation member has a thickness ranging between about 1 mm and about 15 mm.

In yet another aspect of the present invention, the first dielectric material layer comprises a material selected from a group consisting of ceramic, quartz, glass, Teflon, silicone, plastic steel, PP, PC, PE, PET and ABS.

In yet another aspect of the present invention, a potential difference between the discharge electrode and the ground electrode ranges between about 2 k V and about 4.5 k V.

In yet another aspect of the present invention, the first dielectric material layer has a thickness ranging between about 0.1 mm and about 2 mm.

In yet another aspect of the present invention, the device further comprises a second dielectric material layer provided on a side of the ground electrode and the side facing toward the user's skin.

In yet another aspect of the present invention, the second dielectric material layer has a thickness raging between about 5 μm and about 200 μm.

In yet another aspect of the present invention, the second dielectric material layer comprises a material selected from a group consisting of ceramic, quartz, glass, Teflon, silicone, plastic steel, PP, PC, PE, PET and ABS.

In yet another aspect of the present invention, the first dielectric material layer has a distance with the use's skin, and the distance is less than 2 mm.

In yet another aspect of the present invention, a device for treating a user's skin using plasma is provided. The device comprises a housing, accommodating a plasma generation assembly, and the plasma generation assembly including a discharge electrode, a first dielectric material layer positioned on the discharge electrode, a ground electrode and an insulation member spacing the discharge electrode from the ground electrode; wherein the housing has a head at an upper portion of the housing, and the head comprising an opening; and wherein the opening exposes the first dielectric material layer, the ground electrode and the insulation member.

In yet another aspect of the present invention, a device for treating a user's skin using plasma is provided. The device comprises a housing, accommodating a plasma generation assembly, and the plasma generation assembly including a discharge electrode, a first dielectric material layer positioned on the discharge electrode, a ground electrode, an insulation member spacing the discharge electrode from the ground electrode, and a second dielectric material layer positioned at least on the ground electrode; wherein the housing has a head at an upper portion of the housing, and the head comprising an opening; and wherein the opening at least exposes the first dielectric material layer, and the second dielectric material layer.

### BRIEF DESCRIPTION OF THE DRAWINGS

In order to sufficiently understand the essence, advantages and the preferred embodiments of the present invention, the following detailed description will be more clearly understood by referring to the accompanying drawings.

FIG. 1 depicts a conventional portable plasma skin improvement apparatus, which is captured from FIG. 2 of WO2016/114504A1.

FIG. 2 depicts a conventional skin treating device, which is captured from FIG. 3 of US2018/0126183A1.

FIG. 3 depicts an outward appearance of a portable and compact device for treating user's skin using plasma in accordance with a first embodiment of the present invention.

FIG. 4 depicts internal components of the portable and compact device for treating user's skin using plasma in accordance with the first embodiment of the present invention.

FIGS. 5A and 5B depict a top view and a cross-sectional view showing a part of the structure of the plasma generation assembly of the portable and compact device for treating user's skin using plasma in accordance with the first embodiment of the present invention.

FIG. 6 depicts a diagram showing the relationship between the output mode (also referred to as “Duty cycle”) and the plasma's intensity.

FIG. 7 depicts a diagram showing the relationship between the output mode (Duty cycle) and the power output of plasma.

FIG. 8 depicts an outward appearance of a portable and compact device for treating user's skin using plasma in accordance with a second embodiment of the present invention.

FIGS. 9A and 9B depict a top view and a cross-sectional view showing part of the structure of the plasma generation assembly of the portable and compact device for treating user's skin using plasma in accordance with the second embodiment of the present invention.

FIG. 10 depicts a diagram showing the relationship between the thickness of the second dielectric material layer and the plasma current output.

FIGS. 11A and 11B depict a top view and a cross-sectional view showing part of the structure of the plasma generation assembly of the portable and compact device for treating user's skin using plasma in accordance with a third embodiment of the present invention.

FIG. 12 depicts an outward appearance of a portable and compact device for treating user's skin using plasma in accordance with a fourth embodiment of the present invention.

FIG. 13 depicts internal components of the portable and compact device for treating user's skin using plasma in accordance with the fourth embodiment of the present invention.

FIGS. 14A and 14B depict a top view and a cross-sectional view showing part of the structure of the plasma generation assembly of the portable and compact device for treating user's skin using plasma in accordance with the fourth embodiment of the present invention.

FIG. 14C depicts a cross-sectional view showing part of the structure of the plasma generation assembly of the portable and compact device for treating user's skin using plasma in accordance with another embodiment of the present invention.

FIG. 15 depicts a variation of the first embodiment according to the present disclosure.

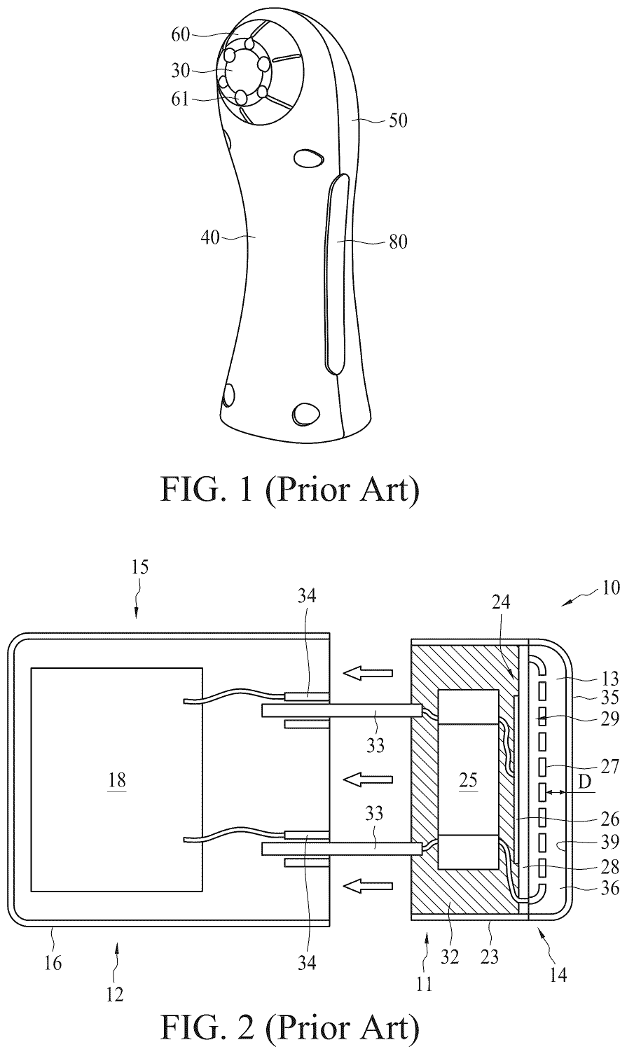

### DETAILED DESCRIPTION OF THE INVENTION

The following description shows the preferred embodiments of the present invention. The present invention is described below by referring to the embodiments and the figures. Thus, the present invention is not intended to be limited to the embodiments shown, but is to be accorded the principles disclosed herein. Furthermore, that various modifications or changes in light thereof will be suggested to persons skilled in the art and are to be included within the spirit and purview of this application and scope of the appended claims.

### The First Embodiment

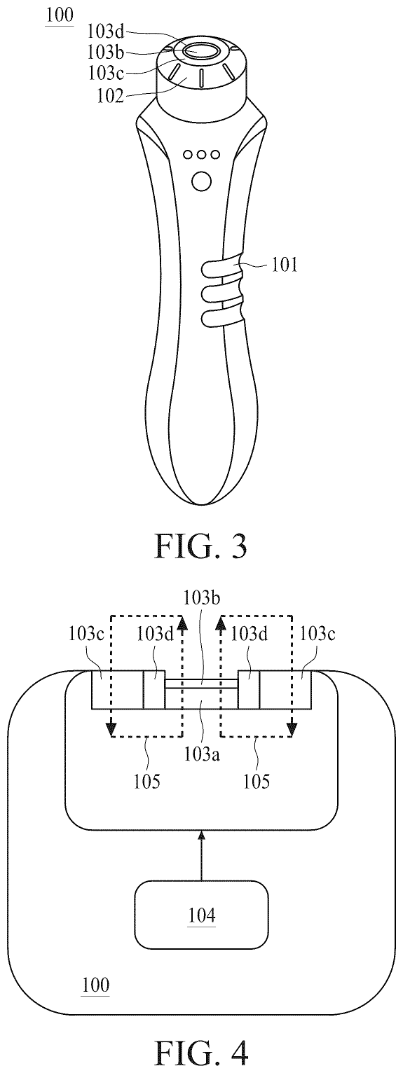

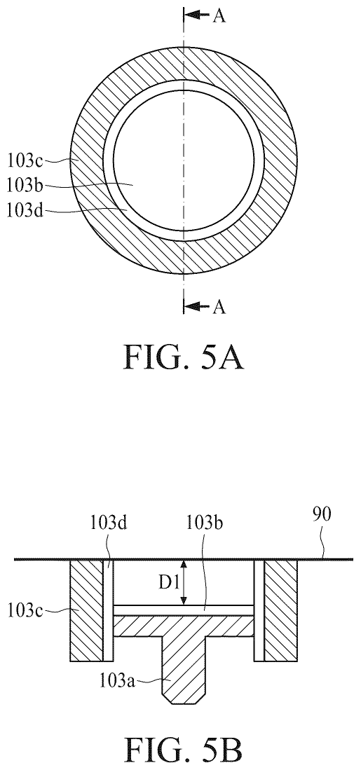

The first embodiment will be described with refer to FIGS. 3, 4 5A, and 5B. FIG. 3 depicts an outward appearance of a portable and compact device (100) for treating user's skin using plasma in accordance with the first embodiment of the present invention. FIG. 4 depicts internal components of the device (100) in accordance with the first embodiment of the present invention. FIGS. 5A and 5B depict a top view showing part of the outward appearance of the plasma generation assembly of the device (100) and a cross-sectional view showing a part of the structure of the plasma generation assembly of the device (100) in accordance with the first embodiment of the present invention along the A-A line.

Firstly, refer to FIGS. 3 and 4. A device (100) for treating a user's skin using plasma includes a housing (101) and a protruding head (102) at the upper portion of the housing (101). A user holds the device (100) and lets the protruding head (102) touch with the user's skin when the device (100) is in use. The device (100) includes a plasma generation assembly (103) and a power supply (104) coupled to the plasma generation assembly (103) in the housing (101).

Further refer to FIGS. 5A and 5B, the plasma generation assembly (103) includes a discharge electrode (103a) having a first surface; a first dielectric material layer (103b) provided on the first surface of the discharge electrode (103a) and the first surface facing toward the user's skin (90); a ground electrode (103c) surrounding the discharge electrode (103a) and being closer to the user's skin (90) than the discharge electrode (103a) when the device is in use; and an insulation member (103d) spacing around the discharge electrode (103a) from the ground electrode (103c). The insulation member (103d) is preferred to have a thickness (a distance spacing the discharge electrode and the ground electrode) ranging between about 1 mm and about 15 mm, and more preferred to a thickness ranging between about 3 mm and about 10 mm in view of insulating property and plasma's intensity. The first dielectric material layer (103b) is preferred to be made by a material selected from a group consisting of ceramic, quartz, glass, Teflon, silicone, plastic steel, PP, PC, PE, PET and ABS and have a thickness ranging between about 0.1 mm and about 2 mm.

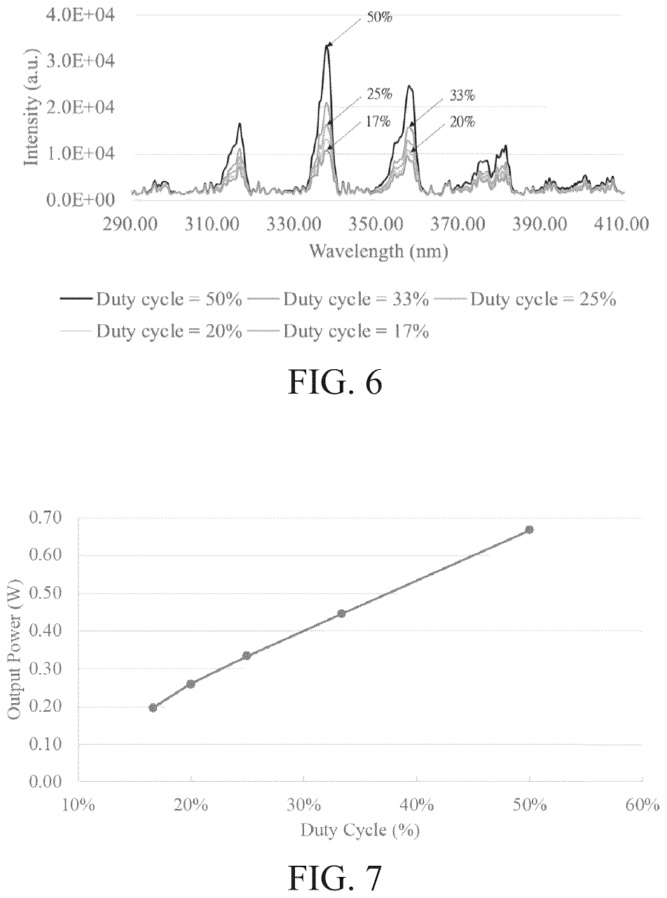

When the device (100) is in use, the power supply (104) powers the plasma generation assembly (103) so as to create a potential difference between the discharge electrode (103a) and the ground electrode (103c) preferably ranging between about 2 k V and about 4.5 k V and more preferably ranging between about 2.5 k V and about 4 k V, so that plasma is generated from the first surface of the discharge electrode (103a) to the ground electrode (103d) and between the first dielectric material layer (103b) and the user's skin (90). The output mode (Duty cycle) of the power supply (104) can be adjusted so that the plasma's intensity and power output can be regulated as shown in FIG. 6 and FIG. 7, and then the suitable plasma current output can be regulated.

The protruding head (102) has a circular opening where the plasma generation assembly (103) can accommodate and exposes the first dielectric material layer (103b), the insulation member (103d) and the ground electrode (103c) to the outside. When the device (100) is in use, the insulation member (103d) and the ground electrode (103c) are in touch with the user's skin. The first dielectric material layer (103b) is preferred to have a distance (D1) (also named as discharge distance) with the use's skin (90), which makes a space formed by the first dielectric material layer (103b), the insulation member (103d) and the user's skin for generation of plasma. D1 is preferred to be less than 2 mm and more preferred to be between about 0.3 mm and about 1.2 mm.

Back to FIG. 4, when the user uses the device (100) to treat the skin of the user's face (or skin), the ground electrode (103c) is in touch with the skin of the user's face. The skin of the user's face can also be deemed as ground, and thus a current loop (105) flowing from the discharge electrode (103a) to the skin of the user's face and the ground electrode (103c) is generated. The current loop (105) is safely limited at the area of the user's face and thus no current would pass through the user's heart. Besides, the intensity of plasma is considerably good because the current loop (105) is directly generated onto the user's skin.

### The Second Embodiment

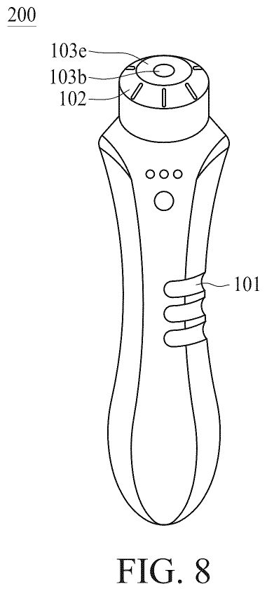

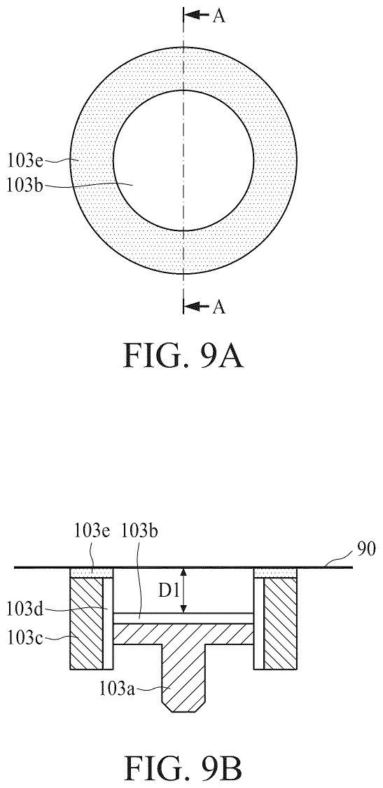

The second embodiment will be described with refer to FIGS. 8 and 9. FIG. 8 depicts an outward appearance of a portable and compact device (200) for treating user's skin using plasma in accordance with the second embodiment of the present invention. FIGS. 9A and 9B respectively depict a top view showing part of the outward appearance of the plasma generation assembly of the device (200) and a cross-sectional view showing a part of the structure of the plasma generation assembly of the device (200) in accordance with the second embodiment of the present invention along the A-A line. The element of the second embodiment which is the same or similar with that of the first embodiment will be given the same or similar symbol.

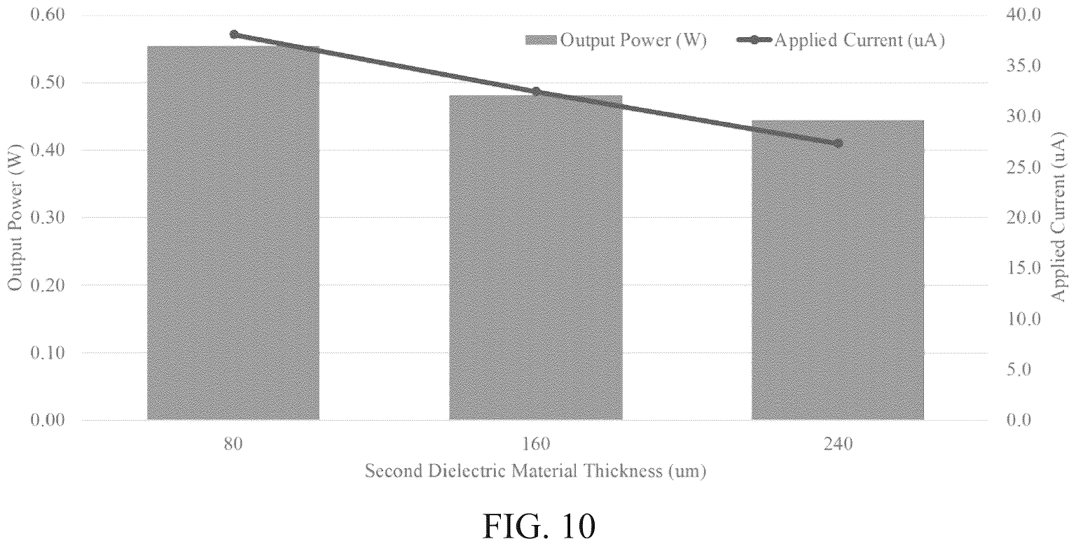

Refer to FIGS. 8 and 9A-9B. A device (200) for treating a user's skin using plasma includes a housing (101) and a protruding head (102) at the upper portion of the housing (101). A user holds the device (200) and lets the protruding head (102) touch with the user's skin when the device (200) is in use. The device (200) includes a plasma generation assembly and a power supply coupled to the plasma generation assembly in the housing (101). The power supply of the device (200) is the same with the power supply (104) described as the first embodiment. The plasma generation assembly of the device (200) is the same with the plasma generation assembly (103) described as the first embodiment except that there is further a second dielectric material layer (103e) provided at least on a side of the ground electrode (103c) and the side facing toward the user's skin (90). Alternatively, the second dielectric material layer (103e) may be both provided on a side of the ground electrode (103c) and a side of the insulation member (103d), and both sides are facing toward the user's skin (90), as shown in FIG. 9B. The second dielectric material layer (103e) is preferred to be made by a material selected from a group consisting of ceramic, quartz, glass, Teflon, silicone, plastic steel, PP, PC, PE, PET and ABS and have a thickness raging between about 5 μm and about 200 μm. It is noted that the user can sense the intensity of the plasma current output. As shown in FIG. 10, the thickness of the second dielectric material layer can be adjusted to regulate the plasma current output. If the output current is too large, it makes the user uncomfortable. However, if the output current is too small, the plasma power is weak and the efficacy of plasma treatment would then be reduced. The suitable thickness of the dielectric material layer is a key factor of balancing user experience and efficacy.

The protruding head (102) has a circular opening where the plasma generation assembly can accommodate and exposes the first dielectric material layer (103b) and the second dielectric material layer (103e) to the outside. When the device (200) is in use, the second dielectric material layer (103e) is in touch with the user's skin (90). The first dielectric material layer (103b) is preferred to have a distance (D1) with the use's skin (90), which makes a space formed by the first dielectric material layer (103b), the insulation member (103d), the second dielectric material layer (103e) and the user's skin (90) for generation of plasma. D1 is preferred to be less than 2 mm and more preferred to be between about 0.3 mm and about 1.2 mm.

When the user uses the device (200) to treat the skin of the user's face, the second dielectric material layer (103e) is in touch with the skin of the user's face. The second dielectric material layer (103e) and the skin of the user's face can also be deemed as ground, and thus a current loop flowing from the discharge electrode (103a) to the second dielectric material layer (103e), the skin of the user's face and the ground electrode (103c) is generated. The current loop is safely limited at the area of the user's face and thus no current would pass through the user's heart. Besides, since the plasma and the current loop are directly generated onto the user's skin (90), the reactive radicals will be directly generated onto and acted on the skin without the problem of retention time. Therefore, the plasma efficacy will be significant.

### The Third Embodiment

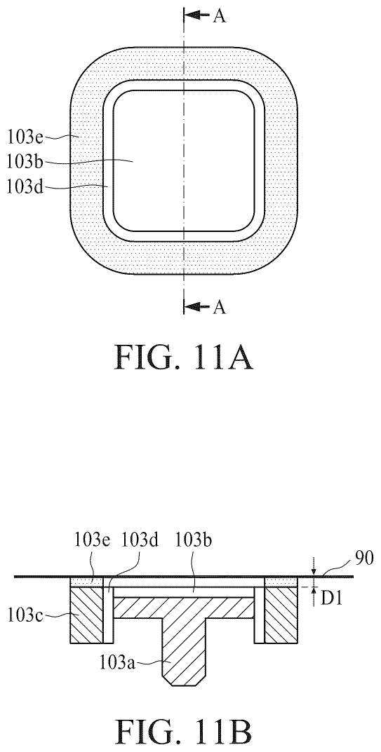

The third embodiment will be described with refer to FIGS. 11A-11B. FIGS. 11A and 11B respectively depict a top view showing part of the outward appearance of the plasma generation assembly of the portable and compact device for treating user's skin using plasma and a cross-sectional view showing part of the structure of the plasma generation assembly of the portable and compact device for treating user's skin using plasma in accordance with the third embodiment of the present invention along the A-A line. The element of the third embodiment which is the same or similar with that of the first or second embodiment will be given the same or similar symbol.

The structure and composition of the portable and compact device for treating user's skin using plasma in accordance with the third embodiment of the present invention is the same with that of the device (100) or the device (200) in the second embodiment except D1 and that the protruding head has a square opening where the plasma generation assembly can correspondingly accommodate and exposes the first dielectric material layer (103b), the insulation member (103d) and the ground electrode (103c) as shown in FIGS. 3, 4 and 5 or exposes the first dielectric material layer (103b) and the second dielectric material layer (103e) as shown in FIGS. 8 and 9A-9B to the outside.

The same description made to the same element is omitted here for simplicity.

One should note that the shape of the protruding head and/or the shape of the opening of the protruding head is not necessary to be the same or similar and can be varied based on the demand and design of the portable and compact device for treating user's skin using plasma. For example, the shape of the protruding head can be formed as an oval, rectangle, polygon, star and the shape of the opening of the protruding head can be formed as diamond, and so on, or the shape of the protruding head can be formed as a round and the shape of the opening of the protruding head can be formed as an oval, rectangle, polygon, star, diamond, and so on.

### The Fourth Embodiment

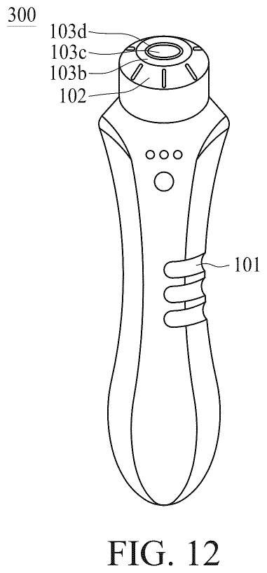

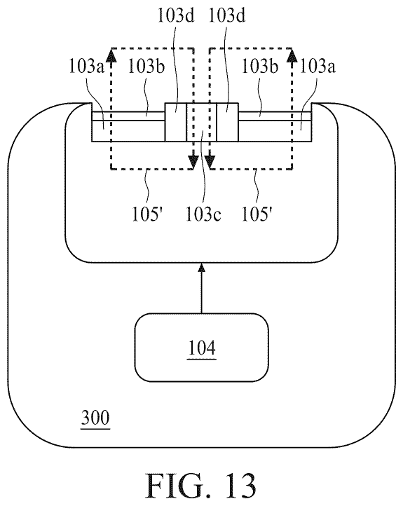

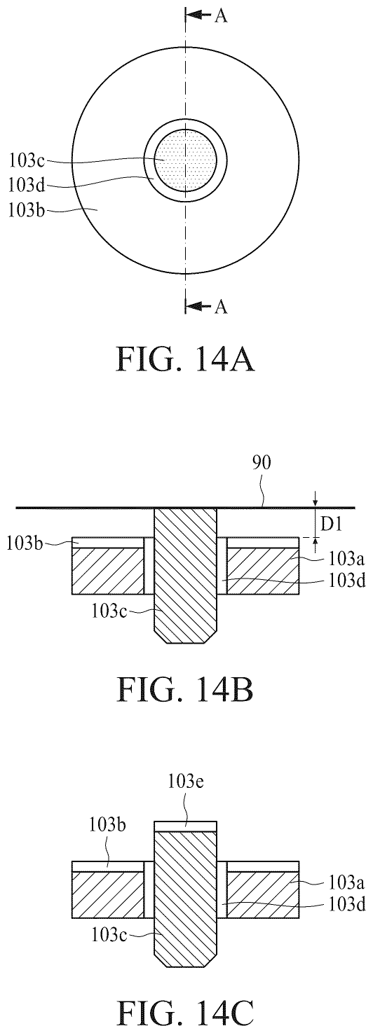

The fourth embodiment will be described with refer to FIGS. 12, 13 and 14A-14C. FIG. 12 depicts an outward appearance of a portable and compact device (300) for treating user's skin using plasma in accordance with the fourth embodiment of the present invention. FIG. 13 depicts internal components of the portable and compact device for treating user's skin using plasma in accordance with the fourth embodiment of the present invention. FIGS. 14A-14B respectively depicts a top view and a sectional view showing part of the structure of the plasma generation assembly of the portable and compact device (300) for treating user's skin using plasma in accordance with the fourth embodiment of the present invention. The element of the fourth embodiment which is the same or similar with that of the first, second or third embodiment will be given the same or similar symbol.

Refer to FIGS. 12 and 13. A device (300) for treating a user's skin using plasma includes a housing (101) and a protruding head (102) at the upper portion of the housing (101). A user holds the device (300) and lets the protruding head (102) touch with the user's skin when the device (300) is in use. The device (300) includes a plasma generation assembly (103) and a power supply (104) coupled to the plasma generation assembly (103) in the housing (101). The plasma generation assembly (103) of the device (300) is the same with the plasma generation assembly (103) described as the first embodiment except that the discharge electrode (103a) surrounds the ground electrode (103c). The discharge electrode (103a) is farther from the user's skin than the ground electrode (103c) when the device (300) is in use.

Further refer to FIGS. 14A-14C, the protruding head (102) has a circular opening where the plasma generation assembly (103) can be disposed and exposes the first dielectric material layer (103b), the insulation member (103d) and the ground electrode (103c) to the outside. When the device (100) is in use, the ground electrode (103c) is in touch with the user's skin. The first dielectric material layer (103b) is preferred to have a distance (D1) with the use's skin, which makes a space formed by the first dielectric material layer (103b), the insulation member (103d) and the user's skin for generation of plasma. D1 is preferred to be less than 2 mm and more preferred to be between about 0.3 mm and about 1.2 mm.

Back to FIG. 13, when the user uses the device (300) to treat the skin of the user's face, the ground electrode (103c) is in touch with the skin of the user's face. The skin of the user's face can also be deemed as ground, and thus a current loop (105′) flowing from the discharge electrode (103a) to the skin of the user's face and the ground electrode (103c) is generated. The current loop (105′) is safely limited at the area of the user's face and thus no current would pass through the user's heart. Besides, since the plasma and the current loop (105′) are directly generated onto the user's skin, the reactive radicals will be directly generated onto and acted on the skin without the problem of retention time. Therefore, the plasma efficacy will be significant. Comparing to the current loop (105) of the first embodiment, one can be seen that the direction of current flow (while the direction is different) is restricted to the treated region and does not flow to other positions (such as the heart) and it is then safe for use because the position of the discharge electrode (103a) and that of the ground electrode (103c) are swapped.

One should note that a second dielectric material layer may be provided at least on a side of the ground electrode (103c) and the side facing toward the user's skin as well as the second embodiment as shown in FIG. 14C. Alternatively, the second dielectric material layer (103e) may be both provided on a side of the ground electrode (103c) and a side of the insulation member (103d), and both sides are facing toward the user's skin as well as the second embodiment.

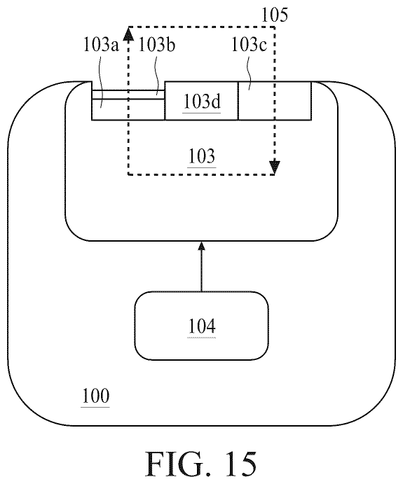

The preferred embodiments of the present invention have been described above. However, those having ordinary skill in the art readily recognize that the disclosure described above can be utilized in a variety of devices, environments, and situations. For example, a ground electrode is not necessarily to surround a discharge electrode, and vice versa. The ground electrode and the discharge electrode can be designed to be adjacent with each other (See FIG. 15) and spaced by an insulation member.

Although the present invention is written with respect to specific embodiments and implementations, various changes and modifications may be suggested to a person having ordinary skill in the art. It is intended that the present disclosure encompass such changes and modifications that fall within the scope of the appended claims.

What is claimed is:

1. A device for treating a user's skin using plasma, comprising:
a plasma generation assembly, comprising:
a discharge electrode including a first surface;
a first dielectric material layer provided on the first surface of the discharge electrode and the first surface facing toward the user's skin;
a ground electrode surrounding the discharge electrode and being closer to the user's skin than the discharge electrode when the device is in use; and
an insulation member spacing around the discharge electrode from the ground electrode; and
a power supply configured to apply power to the plasma generation assembly so that plasma is generated from the first surface of the discharge electrode to the ground electrode and between the first dielectric material layer and the user's skin.

2. The device according to claim 1, wherein the insulation member has a thickness ranging between about 1 mm and about 15 mm.

3. The device according to claim 1, wherein the first dielectric material layer comprises a material selected from a group consisting of ceramic, quartz, glass, Teflon, silicone, plastic steel, PP, PC, PE, PET and ABS.

4. The device according to claim 1, wherein a potential difference between the discharge electrode and the ground electrode ranges between about 2 k V and about 4.5 k V.

5. The device according to claim 1, wherein the first dielectric material layer has a thickness ranging between about 0.1 mm and about 2 mm.

6. The device according to claim 1, further comprising a second dielectric material layer provided on a side of the ground electrode and the side facing toward the user's skin.

7. The device according to claim 6, wherein the second dielectric material layer has a thickness raging between about 5 μm and about 200 μm.

8. The device according to claim 7, wherein the second dielectric material layer comprises a material selected from a group consisting of ceramic, quartz, glass, Teflon, silicone, plastic steel, PP, PC, PE, PET and ABS.

9. The device according to claim 1, wherein the first dielectric material layer has a distance with the use's skin, and the distance is less than 2 mm.

10. A device for treating a user's skin using plasma, comprising:
a plasma generation assembly, comprising:
a ground electrode;
a discharge electrode surrounding the ground electrode and being farther from the user's skin than the ground electrode when the device is in use;
a first dielectric material layer provided on a first surface of the discharge electrode and the first surface facing toward the user's skin; and
an insulation member spacing around the ground electrode from the discharge electrode; and
a power supply configured to apply power to the plasma generation assembly so that plasma is generated from the first surface of the discharge electrode to the ground electrode and between the first dielectric material layer and the user's skin.

11. The device according to claim 10, wherein the insulation member has a thickness ranging between about 1 mm and about 15 mm.

12. The device according to claim 10, wherein the first dielectric material layer comprises a material selected from a group consisting of ceramic, quartz, glass, Teflon, silicone, plastic steel, PP, PC, PE, PET and ABS.

13. The device according to claim 10, wherein a potential difference between the discharge electrode and the ground electrode ranges between about 2 k V and about 4.5 k V.

14. The device according to claim 10, wherein the first dielectric material layer has a thickness ranging between about 0.1 mm and about 2 mm.

15. The device according to claim 10, further comprising a second dielectric material layer provided on a side of the ground electrode and the side facing toward the user's skin.

16. The device according to claim 15, wherein the second dielectric material layer has a thickness raging between about 5 μm and about 200 μm.

17. The device according to claim 15, wherein the second dielectric material layer comprises a material selected from a group consisting of ceramic, quartz, glass, Teflon, silicone, plastic steel, PP, PC, PE, PET and ABS.

18. The device according to claim 10, wherein the first dielectric material layer has a distance with the use's skin, and the distance is less than 2 mm.

19. A device for treating a user's skin using plasma, comprising:
a plasma generation assembly, comprising:
a discharge electrode;
a first dielectric material layer provided on a first surface of the discharge electrode and the first surface facing toward a user's skin;
a ground electrode adjacent to the discharge electrode and being closer to the user's skin than the discharge electrode when the device is in use; and
an insulation member spacing the discharge electrode from the ground electrode; and
a power supply configured to apply power to the plasma generation assembly so that plasma can be generated from the first surface of the discharge electrode to the ground electrode and between the first dielectric material layer and the user's skin.

20. The device according to claim 19, wherein the insulation member has a thickness ranging between about 1 mm and about 15 mm.

21. The device according to claim 19, wherein the first dielectric material layer comprises a material selected from a group consisting of ceramic, quartz, glass, Teflon, silicone, plastic steel, PP, PC, PE, PET and ABS.

22. The device according to claim 19, wherein a potential difference between the discharge electrode and the ground electrode ranges between about 2 k V and about 4.5 k V.

23. The device according to claim 19, wherein the first dielectric material layer has a thickness ranging between about 0.1 mm and about 2 mm.

24. The device according to claim 19, further comprising a second dielectric material layer provided on a side of the ground electrode and the side facing toward the user's skin.

25. The device according to claim 24, wherein the second dielectric material layer has a thickness raging between about 5 μm and about 200 μm.

26. The device according to claim 24, wherein the second dielectric material layer comprises a material selected from a group consisting of ceramic, quartz, glass, Teflon, silicone, plastic steel, PP, PC, PE, PET and ABS.

27. The device according to claim 19, wherein the first dielectric material layer has a distance with the use's skin, and the distance is less than 2 mm.

28. A device for treating a user's skin using plasma, comprising:
a housing, accommodating a plasma generation assembly, and the plasma generation assembly including a discharge electrode, a first dielectric material layer positioned on the discharge electrode, a ground electrode and an insulation member spacing the discharge electrode from the ground electrode;
wherein the housing has a head at an upper portion of the housing, and the head comprising an opening; and
wherein the opening exposes the first dielectric material layer, the ground electrode and the insulation member.
